(19) United States
(12) Patent Application Publication
(10) Pub. No.: US 2025/0064545 A1
(43) Pub. Date: Feb. 27, 2025

(54) TEMPLATE DEVICES FOR PLACEMENT OF IMPLANT AND METHODS OF USE

(71) Applicant: DUKE UNIVERSITY, Durham, NC (US)

(72) Inventors: Brett Phillips, Durham, NC (US); Muath Bishawi, Durham, NC (US); Kristen Rezak, Durham, NC (US); Jess Rames, Durham, NC (US)

(21) Appl. No.: 18/721,773

(22) PCT Filed: Dec. 21, 2022
(86) PCT No.: PCT/US2022/053665

§ 371 Date: Jun. 19, 2024

Related U.S. Application Data
(60) Provisional application No. 63/292,007, filed on Dec. 21, 2021.

(51) Int. Cl. A61B 90/00; A61F 2/12

(52) U.S. Cl. CPC A61B 90/02; A61F 2/12; A61B 2090/3908; A61F 2220/0008; A61F 2230/005

(57) ABSTRACT

The present disclosure provides devices, systems, and methods relating to performing a medical procedure. In particular, the present disclosure is directed to devices, systems, and methods for accurately positioning and securing a body implant in a subject. A template corresponds to the implant, and the template is configured to mark a desired location in a patient prior to positioning the implant at the desired location.

### RELATED APPLICATIONS

This application claims priority to and the benefit of U.S. Provisional Patent Application No. 63/292,007 filed Dec. 21, 2021, which is incorporated herein by reference in its entirety for all purposes.

### FIELD

This present disclosure provides devices, systems, and methods relating to performing a medical procedure. In particular, the present disclosure is directed to devices, systems, and methods for accurately positioning and securing a body implant in a subject.

### BACKGROUND

There are several types of breast reconstruction methods including the use of a tissue expander (TE), as well as saline and silicone filled breast implants. A tissue expander is a type of implant (e.g., a temporary breast implant) that uses the growth of a patient's skin in the reconstruction process. Tissue expanders have tabs or anchors that are used to secure the device to the chest muscle and chest wall. Each type of tissue expander has different locations and measurements of where the tabs are located. In other words, tissue expanders from different manufacturers have different layouts and/or shapes (e.g., round, teardrop, etc.). The knowledge of where to secure the tabs depends on the surgeon's experience and the placement is roughly estimated before implanting the sterile tissue expander.

It is important that the implant is positioned under the skin and at the correct muscle attachment points in order to obtain the desired shape and size to achieve symmetry. For example, positioning of the tissue expander is important, but challenging because of the need to use aseptic technique and minimize touching of the implant. When using a minimal touch or one touch technique in reconstructive surgery, the tissue expander device itself is not placed into the breast defect until it is ready to be implanted. Therefore, there is a need for a template to help guide the accurate placement of the tissue expander on the chest wall before final sterile placement. Current procedures do not use a guide and the tab securement of the tissue expander is done with less accuracy. The ability to precisely place a tissue expander on the first attempt may reduce the number of complications.

Similar problems exist with planning placement exist for breast implants, and other body implants (temporary or permanent). Currently approved FDA permanent breast implants do not include any methods for fixation (e.g., tabs). WO2021/150917, incorporated herein by reference in its entirety, discloses an implant with biocompatible and biodegradable materials to provide enhanced long-term fixation of the implant. Therefore, there is also a need for a template to help guide the accurate placement of various breast implants before final sterile placement.

### SUMMARY

The Summary is provided to introduce a selection of concepts that are further described below in the Detailed Description. This Summary is not intended to identify key or essential features of the claimed subject matter, nor is it intended to be used as an aid in limiting the scope of the claimed subject matter.

One aspect of the present disclosure provides a measurement template for placement of tissue expanders and implants in reconstructive and aesthetic surgery. The present disclosure details a sterile, disposable, and foldable template that is unique to every type of tabbed tissue expander in order to help guide the placement in surgery.

In one aspect, the present disclosure provides a device for positioning a tissue expander in a patient. The device comprises a body: a first projection extending from the body; a second projection extending from the body: a first aperture positioned on the first projection; and a second aperture positioned on the second projection. The second aperture is positioned a first distance from the first aperture and the first distance is equal to a second distance between attachment points on the tissue expander.

In some embodiments, the device further includes a third projection extending from the body and a third aperture position on the third projection.

In some embodiments, the second projection is positioned between the first projection and the third projection.

In some embodiments, the third aperture is positioned a second distance from the second aperture.

In some embodiments, the second distance is larger than the first distance.

In some embodiments, the second distance is equal to the first distance.

In some embodiments, the device further includes a plurality of projections including the first projection and the second projection, and further comprising a plurality of apertures including the first aperture and the second aperture. Each of the plurality of projections includes at least one of the plurality of apertures.

In some embodiments, the plurality of apertures is equally spaced circumferentially around the body.

In some embodiments, the plurality of apertures is unequally spaced circumferentially around the body.

In some embodiments, the device is star shaped.

In some embodiments, the body includes a body aperture, and the body aperture is positioned at a geometric center of the device.

In some embodiments, the device is flexible.

In some embodiments, the device is configured to fold for insertion through an incision in the patient and configured to conform to the contours of a chest wall of the patient.

In some embodiments, the device is silicone.

In some embodiments, the device is sterile, disposable, and foldable.

In one aspect, the present disclosure provides a device for positioning a breast implant at a desired location in a patient. The device comprises a body having a periphery corresponding to an outer periphery of the breast implant; and an aperture formed in the body. The aperture is positioned at a geometric center of the device. The device is configured to be positioned at the desired location and the periphery is marked to identify the desired location for the breast implant.

In some embodiments, the periphery is an outer-most edge of the device.

In some embodiments, the periphery is an edge spaced radially inward from an outer-most edge of the device.

In some embodiments, the body is trimmable to adjust the periphery of the body.

In some embodiments, the body is circular.

In some embodiments, the device is sterile, disposable, and foldable.

In some embodiments, the body further comprising a fiducial marking to indicate an orientation of the device.

In some embodiments, the fiducial marking is tactile.

In some embodiments, the body includes a radially inward portion, a radially outward portion, and a strut extending between the radially inward portion and the radially outward portion.

In some embodiments, the body further includes a slot at least partially formed between the radially inward portion, the radially outward portion, and the strut.

In some embodiments, the periphery is at least partially defined by the slot.

In some embodiments, the body further includes a plurality of circumferential slots.

In some embodiments, the body has a thickness within a range of 2 cm and 6 cm.

In some embodiments, the body further includes a second periphery corresponding to an outer periphery of a second breast implant.

In one aspect, the present disclosure provides a kit comprising an implant and a template corresponding to the implant. The template is configured to mark a desired location in a patient prior to positioning the implant at the desired location.

In some embodiments, the kit further includes a marker.

In some embodiments, the implant is a tissue expander or a breast implant.

In some embodiments, the kit further includes a biodegradable attachment means for securing the implant at the desired location.

In one aspect, the present disclosure provides a method of marking a desired location for an implant in a patient. The method comprising: making an incision in the patient: inserting a template through the incision; and positioning the template in the desired location in the patient. The method further comprises marking the desired location; and removing the template through the incision.

In some embodiments, the method further comprises inserting an implant through the incision and positioning the implant at the desired location.

In some embodiments, the method further comprises attaching the implant to the patient at the desired location.

In some embodiments, the method further comprises discarding the template.

In some embodiments, inserting the template through the incision includes folding the template to fit through the incision.

In some embodiments, marking the desired location includes tracing a portion of the template with a marker.

### BRIEF DESCRIPTION OF THE DRAWINGS

The accompanying figures are provided by way of illustration and not by way of limitation.

FIG. 1A is a perspective view of a template device for a tissue expander.

FIG. 1B is a front view of the template device of FIG. 1A.

FIG. 1C is a bottom view of the template device of FIG. 1A.

FIG. 1D is a front view of a tissue expander with tab attachment points.

FIG. 2A is a perspective view of a template device for a tissue expander.

FIG. 2B is a front view of a tissue expander with tab attachment points.

FIG. 3 is a front view of a template device for a tissue expander.

FIG. 4A is a perspective view of a template device for a breast implant.

FIG. 4B is a front view of the template device of FIG. 4A.

FIG. 4C is a bottom view of the template device of FIG. 4A.

FIG. 5A is a perspective view of a template device for a breast implant.

FIG. 5B is a front view of the template device of FIG. 5A.

FIG. 5C is a bottom view of the template device of FIG. 5A.

FIG. 6 is a front view of a template device for a breast implant.

FIG. 7 is a flowchart of a method for marking a desired location for an implant in a patient.

### DETAILED DESCRIPTION

Section headings as used in this section and the entire disclosure herein are merely for organizational purposes and are not intended to be limiting.

All publications, patent applications, patents and other references mentioned herein are incorporated by reference in their entirety.

### 1. Definitions

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present disclosure. All publications, patent applications, patents and other references mentioned herein are incorporated by reference in their entirety. The materials, methods, and examples disclosed herein are illustrative only and not intended to be limiting.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “and” and “the” include plural references unless the context clearly dictates otherwise. The present disclosure also contemplates other embodiments “comprising,” “consisting of” and “consisting essentially of,” the embodiments or elements presented herein, whether explicitly set forth or not.

As used herein, the transitional phrase “consisting essentially of” (and grammatical variants) is to be interpreted as encompassing the recited materials or steps “and those that do not materially affect the basic and novel characteristic(s)” of the claimed invention. Thus, the term “consisting essentially of” as used herein should not be interpreted as equivalent to “comprising.”

“About” is used to provide flexibility to a numerical range endpoint by providing that a given value may be “slightly above” or “slightly below” the endpoint without affecting the desired result.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated. Recitation of ranges of values herein are merely intended to serve as a shorthand method of referring individually to each separate value falling within the range, unless otherwise-Indicated herein, and each separate value is incorporated into the specification as if it were individually recited herein. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this disclosure.

“Subject” and “patient” as used herein interchangeably refers to any vertebrate, including, but not limited to, a mammal (e.g., cow; pig, camel, llama, horse, goat, rabbit, sheep, hamsters, guinea pig, cat, dog, rat, and mouse, a non-human primate (e.g., a monkey, such as a cynomolgus or rhesus monkey, chimpanzee, etc.) and a human). In some embodiments, the subject may be a human or a non-human. In one embodiment, the subject is a human. The subject or patient may be undergoing various forms of treatment.

The terms “biocompatible” and “biocompatibility” as used herein generally refer to a material or a property of a material that does not cause an adverse reaction in a subject when placed in proximity to a portion of the subject's body or when in contact with one or more of the subject's tissues (internally or externally positioned). Adverse reactions include inflammation, infection, fibrotic tissue formation, cell death, thrombosis, and the like.

The terms “biodegradable” and “bioabsorbable” as used herein generally refer to a material or a property of a material that is capable of being broken down (catabolized and/or metabolized, absorbed and/or excreted) inside a subject by any means, without causing or being associated with a significant adverse reaction in a subject. For example, a biodegradable material and a device made of biodegradable material does not persist within a subject's body long-term, but is substantially absorbed and/or broken down by the subject's body in a manner that has no significant detrimental physiological and/or biochemical effects on the subject.

“Implant” and “body implant” generally refer to an article or device that is placed entirely or partially into a subject (e.g., a breast implant, a tissue expander, etc.), for example by a surgical procedure or medical intervention, for any period of time.

Unless otherwise defined herein, scientific and technical terms used in connection with the present disclosure shall have the meanings that are commonly understood by those of ordinary skill in the art. The meaning and scope of the terms should be clear: in the event, however of any latent ambiguity, definitions provided herein take precedent over any dictionary or extrinsic definition. Further, unless otherwise required by context, singular terms shall include pluralities and plural terms shall include the singular.

### 2. Template for Tissue Expander

Embodiments of the present disclosure relate generally to a template device for positioning a body implant in a subject. The template device aids a surgeon in the placement of implants. In some embodiments, the templates are sterile, disposable, and/or foldable. In general, the template device includes an aperture (e.g., a hole, a slot, a notch, etc.) that corresponds to the predetermined mounting point for a given implant. This allows a surgeon to mark a desired location in a patient's body for later attachment of the implant.

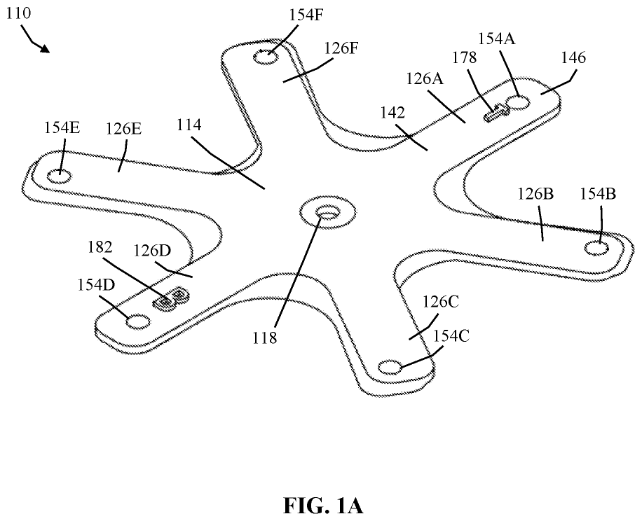

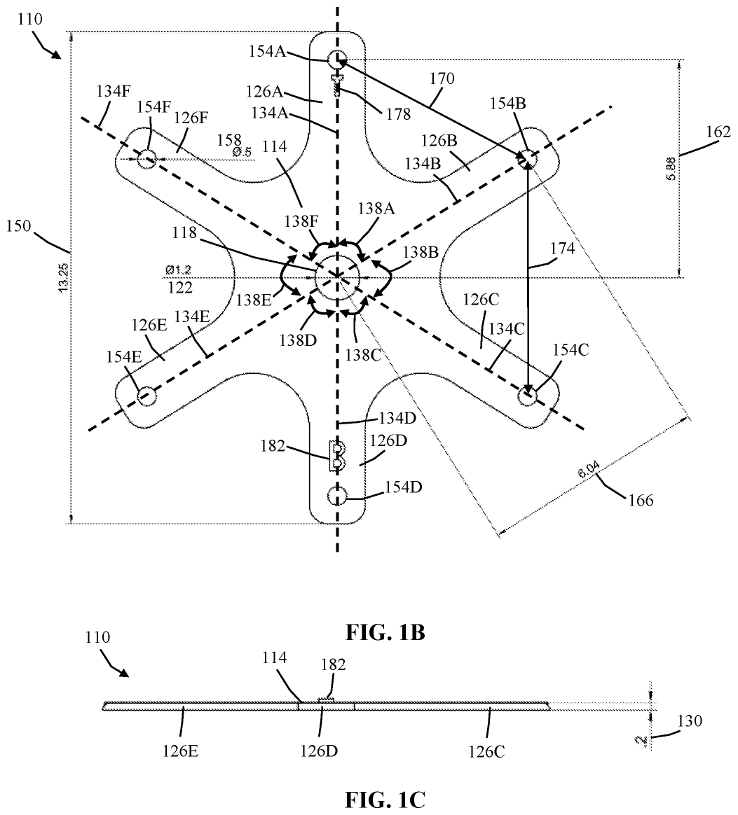

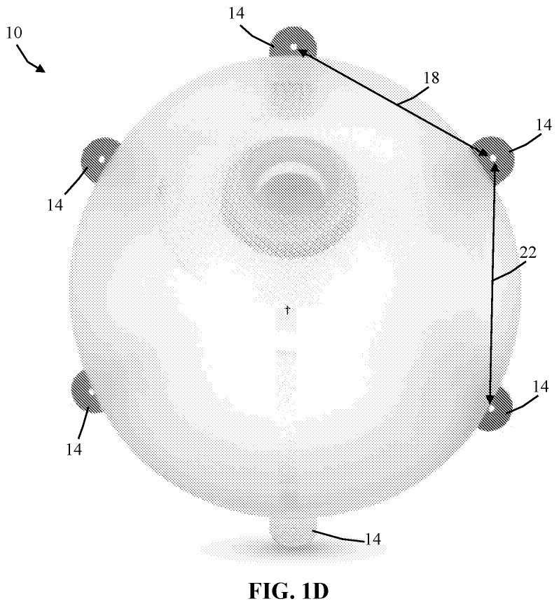

In one embodiment, the template device is designed for positioning a breast tissue expander. Some example embodiments are illustrated in FIGS. 1A-3. Tissue expanders typically have tabs or anchors, which are used to secure the implant to the chest muscle and chest wall. With reference to FIG. 1D, a tissue expander 10 is illustrated and includes a plurality of tabs 14 to secure the tissue expander 10 to the chest muscle and/or chest wall. Each expander company has different types of tissue expanders and implants with various measurements of base width, height, projection and placement of the suture tabs, etc. To add to the difficulty of placement, the tabs may be asymmetric.

With reference to FIGS. 1A-IC, a template device 110 for positioning a tissue expander (e.g., the tissue expander 10) in a patient is illustrated. The device 110 includes a body 114 that is substantially flat. In the illustrated embodiment, the body 114 includes a body aperture 118. In some embodiments, the body aperture 118 is positioned at a geometric center of the device 110 (as viewed from FIG. 1B). In the illustrated embodiment, the body aperture 118 has a diameter 122 of approximately 1.2 cm. In other embodiments, the body aperture 122 has a diameter within a range of approximately 0.5 cm to approximately 2 cm.

A plurality of projections 126A-126F extend from the body 114. In the illustrated embodiment, the plurality of projections 126A-126F includes a first projection 126A, a second projection 126B, a third projection 126C, a fourth projection 126D, a fifth projection 126E, and a sixth projection 126F. In other embodiments, the device includes any number of projections that extend from the body. In the illustrated embodiment, the second projection 126B is positioned circumferentially between the first projection 126A and the third projection 126C. Similarly, the third projection 126C is positioned circumferentially between the second projection 126B and the fourth projection 126D.

With reference to FIG. 1C, the body 114 and the projections 126A-126F have the same thickness dimension 130. In the illustrated embodiment, the thickness 130 is approximately 0.2 cm. In other embodiments, the thickness is within a range of approximately 0.1 cm to approximately 0.5 cm. In other embodiments, the body and the projections have different or varying thicknesses.

In some embodiments, the template devices of the present disclosure exhibit a certain thickness. In some embodiments, the template devices of the present disclosure have a uniform thickness. In other embodiments, the template devices of the present disclosure have varying degrees of thickness.

With reference to FIG. 1B, each of the projections 126A-126F extend along a corresponding projection axis 134A-134F that intersects the body 114. In the illustrated embodiment, each of the projection axes 134A-134F intersects the body aperture 118. Angles 138A-138F are defined between adjacent projection axes 134A-134F. For example, the angle 138A is defined between the first projection axis 134A and the second projection axis 134B. The angle 138B is defined between the second projection axis 134B and the third projection axis 134C. The angle 138C is defined between the third projection axis 134C and the fourth projection axis 134D. The angle 138D is defined between the fourth projection axis 134D and the fifth projection axis 134E. The angle 138E is defined between the fifth projection axis 134E and the sixth projection axis 134F. The angle 138F is defined between the sixth projection axis 134F and the first projection axis 134A. In some embodiments, the angles 138A-138F are each approximately 60 degrees. In the illustrated embodiment, the angles 138A, 138C, 138D, and 138F are each approximately 58 degrees. In the illustrated embodiment, the angles 138B and 138E are each approximately 64 degrees.

In some embodiment, the angles 138A-138F are equal. In other words, the projections 126A-126F are equally spaced circumferentially around the body 114. In other embodiments, the angles 138A-138F are unequal and the projections 134A-134F are unequally spaced circumferentially around the body 114. In other embodiments, the angle between adjacent projection axes is within a range of approximately 5 degrees and 180 approximately 180 degrees.

In the illustrated embodiment, the projections 126A-126F extend radially outward from the body 114. Each of the projections includes a radially inward end portion 142 and a radially outward end portion 146. In the illustrated embodiment, the radially inward end portion 146 is connected to the body 114. In the illustrated embodiment, the device 110 is star shaped (e.g., a 6-pointed star shaped). In other embodiments, the device is star shaped with any number of points.

In the illustrated embodiment, the device 110 has an outer-most dimension 150 of approximately 13.25 cm. In other embodiments, the dimension 150 is within a range of approximately 8 cm to approximately 18 cm. In the illustrated embodiment, the outer-most dimension 150 is measured between the radially outward end portion 146 of the first projection 126A and the radially outward portion 146 of the fourth projection 126D.

The device 110 further includes a plurality of apertures 154A-154F positioned on the projections 126A-126F. In the illustrated embodiment, the plurality of apertures 154A-154F includes a first aperture 154A, a second aperture 154B, a third aperture 154C, a fourth aperture 154D, a fifth aperture 154E, and a sixth aperture 154F. In other embodiments, the device includes any number of apertures positioned on the projections. In the illustrated embodiment, the first aperture 154A is positioned on the first projection 126A, the second aperture 154B is positioned on the second projection 126B, the third aperture 154C is positioned on the third projection 126C, the fourth aperture 154D is positioned on the fourth projection 126D, the fifth aperture 154E is positioned on the fifth projection 126D, and the sixth aperture 154F is positioned on the sixth aperture 126F. In some embodiments, the apertures 154A-154F are equally spaced circumferentially around the body 114. In other embodiments, the apertures 154A-154F are unequally spaced circumferentially around the body 114. In the illustrated embodiment, each of the projections includes a single aperture positioned at the radially outward end portion. In some embodiments, each of the projections includes two apertures positioned at different radial locations. The template apertures can be provided in any suitable manner, such as at the end of the projections that extend from a central body are of the template (e.g., in a star or asterisk shape). Alternately, the aperture positions can be created in any other suitable profile shape that results in a prescribed hole spacing (e.g., polygon, dog bone, anchor, arrow, snowflake, etc., or any combination thereof).

In the illustrated embodiment, the apertures 154A-154F are circular through-hole. In other embodiments, the apertures 154A-154F are any suitable shaped opening (e.g., an oval, a slot, a notch, etc.). In the illustrated embodiment, the apertures 154A-154F have a diameter 158 of approximately 0.5 cm. In other embodiments, the apertures 154A-154F have a diameter within a range of approximately 0.1 cm to approximately 1 cm.

With continued reference to FIG. 1B, the first aperture 154A is positioned a distance 162 from the body aperture 118. The second aperture 154B is positioned a distance 166 from the body aperture 118. In the illustrated embodiment, the distance 166 is larger than the distance 162. In the illustrated embodiment, the second aperture 154B is positioned a distance 170 from the first aperture 154A. The distance 170 is equal to a distance 18 (FIG. 1D) between two adjacent attachment points 14 on the tissue expander 10. In other words, the relative positioning of the first aperture 154A and the second aperture 154B correspond to the relative positioning of the first and second tabs 14 on the tissue expander 10. Likewise, the third aperture 154C is positioned a distance 174 from the second aperture 154B, and the distance 174 corresponds to a distance 22 (FIG. 1D) between two adjacent attachment points 14 on the tissue expander 10. In other words, the relative position of the plurality of apertures 154A-154F correspond to the relative position of the tabs 14 on the tissue expander 10. As such, the apertures 154A-154F provide a template for the attachment points of the tissue expander 10. In some embodiments the distance 174 is larger than the distance 170. In other embodiments, the distances between adjacent apertures are equal.

In some embodiments, the template devices of the present disclosure are malleable, so they can be configured into any desired size and shape. In the illustrated embodiment, the device 110 is flexible. For example, the device 110 is configured to fold over and/or on top of itself for insertion through an incision in the patient. In other word, the device 110 can be folded to reduce an overall dimension (e.g., dimension 150) of the device 110 for insertion through an incision. Furthermore, the device 110 is flexible such that the device 110 is configured to conform to the contours of a chest wall of the patient, which is not completely flat.

In some embodiments, the template device (e.g., device 110) is made of silicone. In some embodiments, the template device is made from a rigid material, a semi-rigid material, or a flexible material. Some non-limiting example materials include paper, biocompatible plastic, mesh, or silicone. As detailed herein, a flexible material such as silicone can advantageously be rolled or folded in order to be inserted through a smaller incision, and it can also conform to the contours of the chest wall. In some embodiments, the template devices disclosed herein are made with additive manufacturing methods (e.g., 3D printed).

The tissue expander template device 110 comprises a substantially flat body 114 with through-holes 154A-154F that correspond to the location of the attachment features on the particular tissue expander that is to be inserted in a subject. Therefore, the template device 110 is provided as a customized design that is specific to a particular size and model of tissue expander. The device 110 is a sterile, disposable, and foldable template that is placed on a patient prior to implantation to guide the positioning of the tissue expander and reduce the possibility of incorrect placement of the tissue expander. Specifically, the template device 110 helps align the tabs and anchors of a given tissue expander that attach to the chest muscle and/or chest wall.

Template devices, in some embodiments, include ergonomic features (e.g., ribs, ridges, bosses, etc. for case of handling) and fiducial markings. The markings can be tactile (e.g., raised or embossed) to improve blind placement. In the illustrated embodiment, the device 110 includes a first marking 178 on the first projection 126A of a raised “T” and a second marking 182 on the fourth projection 126D of a raised “B”.

In some embodiments, the template device is substantially coated with at least one biologically active agent. In some embodiments, the biologically active agent includes, but is not limited to, an antibacterial agent, an antifungal agent, an anti-inflammatory agent, and any combinations thereof. In some embodiments, the biologically active agent imparts to the device an advantageous property (e.g., sterility). The biologically active agent can be pre-applied to the device, and/or applied as part of a medical procedure by a medical professional.

These and other advantages would be readily apparent to one of ordinary skill in the art based on the present disclosure. It would also be apparent to one of ordinary skill that, although the template devices and systems disclosed herein are primarily described with reference to tissue expanders and breast implants, it is within the scope of the disclosure that the device can be used with many other types of bodily implants. While the template is describe with respect to breast construction, it is envisioned that the template can be adapted to other applications where it is useful to pre-position an implanted device. For example, a similar template device could be used for other implant devices that require precise positioning and limited contact.

It is additionally noted that, although a customized template has some advantages, it is within the scope of the disclosure to include multiple template patterns within a single physical template. Further, it is possible to provide the template such that it can be trimmed or resized.

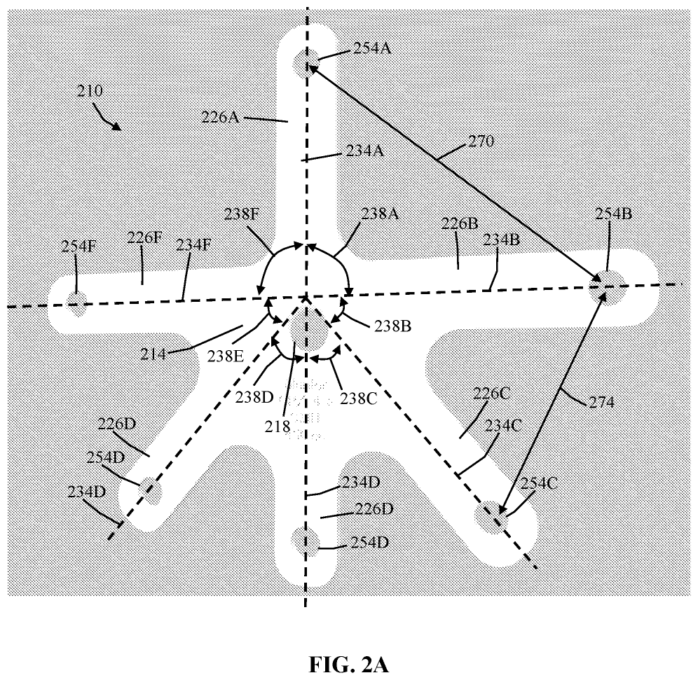

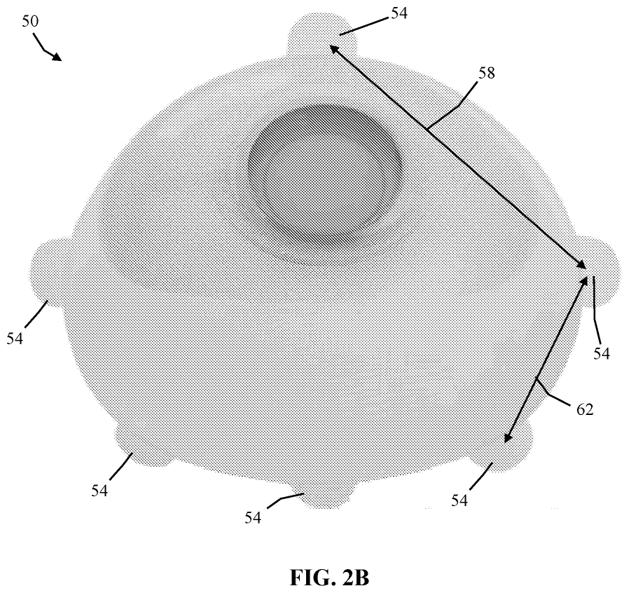

With reference to FIG. 2A, a template device 210 for positioning a tissue expander in a patient is illustrated. The device 210 includes a body 214 that is substantially flat. In the illustrated embodiment, the body 214 includes a body aperture 218. The template device 210 is similar to the template device 110, with similar aspects identified with similar reference numerals incremented by 100.

A plurality of projections 226A-226F extend from the body 214. In the illustrated embodiment, the plurality of projections 226A-226F includes a first projection 226A, a second projection 226B, a third projection 226C, a fourth projection 226D, a fifth projection 226E, and a sixth projection 226F. In the illustrated embodiment, the second projection 226B is positioned circumferentially between the first projection 226A and the third projection 226C. Similarly, the third projection 226C is positioned circumferentially between the second projection 226B and the fourth projection 226D.

With continued reference to FIG. 2A, each of the projections 226A-226F extend along a corresponding projection axis 234A-234F that intersects the body 214. In the illustrated embodiment, some of the projection axes 234A-234F do not intersect the body aperture 241.

Angles 238A-238F are defined between adjacent projection axes 234A-234F. For example, the angle 238A is defined between the first projection axis 234A and the second projection axis 234B. The angle 238B is defined between the second projection axis 234B and the third projection axis 234C. The angle 238C is defined between the third projection axis 234C and the fourth projection axis 234D. The angle 238D is defined between the fourth projection axis 234D and the fifth projection axis 234E. The angle 238E is defined between the fifth projection axis 234E and the sixth projection axis 234F. The angle 238F is defined between the sixth projection axis 234F and the first projection axis 234A. In the illustrated embodiment, the angles 238A and 238F are each approximately 90 degrees, and the angles 238B-238E are each approximately 45 degrees. In the illustrated embodiment, the angles 238A-238F are unequal. In the illustrated embodiment, the projections 226A-226F are unequally spaced circumferentially around the body 214.

The device 210 further includes a plurality of apertures 254A-254F positioned on the projections 226A-226F. In the illustrated embodiment, the plurality of apertures 254A-254F includes a first aperture 254A, a second aperture 254B, a third aperture 254C, a fourth aperture 254D, a fifth aperture 254E, and a sixth aperture 254F. In other embodiments, the device includes any number of apertures positioned on the projections. In the illustrated embodiment, the first aperture 254A is positioned on the first projection 226A, the second aperture 254B is positioned on the second projection 226B, the third aperture 254C is positioned on the third projection 226C, the fourth aperture 254D is positioned on the fourth projection 226D, the fifth aperture 254E is positioned on the fifth projection 226E, and the sixth aperture 254F is positioned on the sixth projection 226F.

With reference to FIG. 2B, a tissue expander 50 is illustrated and includes a plurality of tabs 54 to secure the tissue expander 50 to the chest muscle and/or chest wall.

With continued reference to FIG. 2A, the second aperture 254B is positioned a distance 270 from the first aperture 254A. The distance 270 is equal to a distance 58 (FIG. 2B) between two adjacent attachment points 54 on the tissue expander 50. In other words, the relative positioning of the first aperture 254A and the second aperture 254B correspond to the relative positioning of the first and second tabs 54 on the tissue expander 50. Likewise, the third aperture 254C is positioned a distance 274 from the second aperture 254B, and the distance 274 corresponds to a distance 62 (FIG. 2B) between two adjacent attachment points 54 on the tissue expander 50. In other words, the relative position of the plurality of apertures 254A-254F correspond to the relative position of the tabs 54 on the tissue expander 50. As such, the apertures 254A-254F provide a template for the attachment points of the tissue expander 50. In the illustrated embodiment, the distance 270 is larger than the distance 274.

The tissue expander template device 210 comprises a substantially flat body 214 with through-holes 254A-254F that correspond to the location of the attachment features on the particular tissue expander that is to be inserted in a subject. Therefore, the template device 210 is provided as a customized design that is specific to a particular size and model of tissue expander.

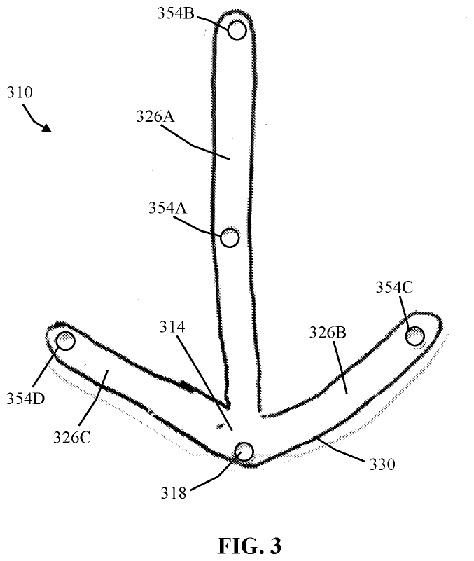

With reference to FIG. 3, a template device 310 for positioning a tissue expander in a patient is illustrated. The device 310 includes a body 314 that is substantially flat. In the illustrated embodiment, the body 314 includes a body aperture 318.

A plurality of projections 326A-326C extend from the body 314. In the illustrated embodiment, the plurality of projections 326A-326C includes a first projection 326A, a second projection 326B, and a third projection 326C. In the illustrated embodiment, the second projection 326B and the third projection 326C are arcuate and form a lower curved edge 330. In some embodiments, the curved edge 330 is used to locate, for example, the inframammary fold (IMF) on a patient.

The device 310 further includes a plurality of apertures 354A-354D formed on the projections 326A-326C. In the illustrated embodiment, the first projection 326A includes a first aperture 354A positioned radially inward and a second aperture 354B positioned radially outward. In other words, the first aperture 354A is positioned between the second aperture 354B and the body aperture 318. The third aperture 354C is positioned on the second projection 326B, and the fourth aperture 354D is positioned on the third projection 326C.

### 3. Template for Breast Implant

In other embodiments, the template device is configured for positioning a breast implant. Unlike tissue expanders, permanent implants typically do not include permanent attachment features. However, some implants, such as those described in PCT Patent Application PCT/US2021/014650, have temporary and biodegradable attachment features to increase placement accuracy. Further, unlike tissue expanders, breast implants are typically exactly circular in shape. Thus, the implant template devices disclosed herein are designed to mark an area (e.g., a circular area) in which the implant should be placed.

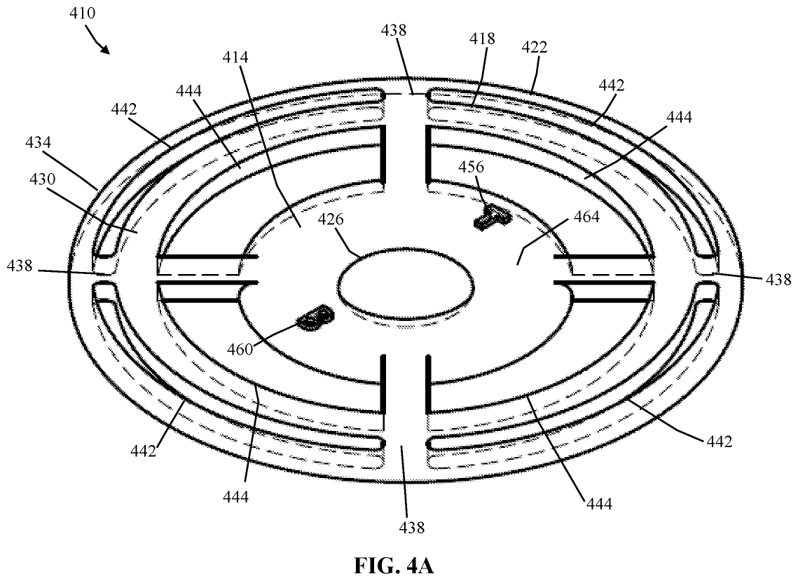

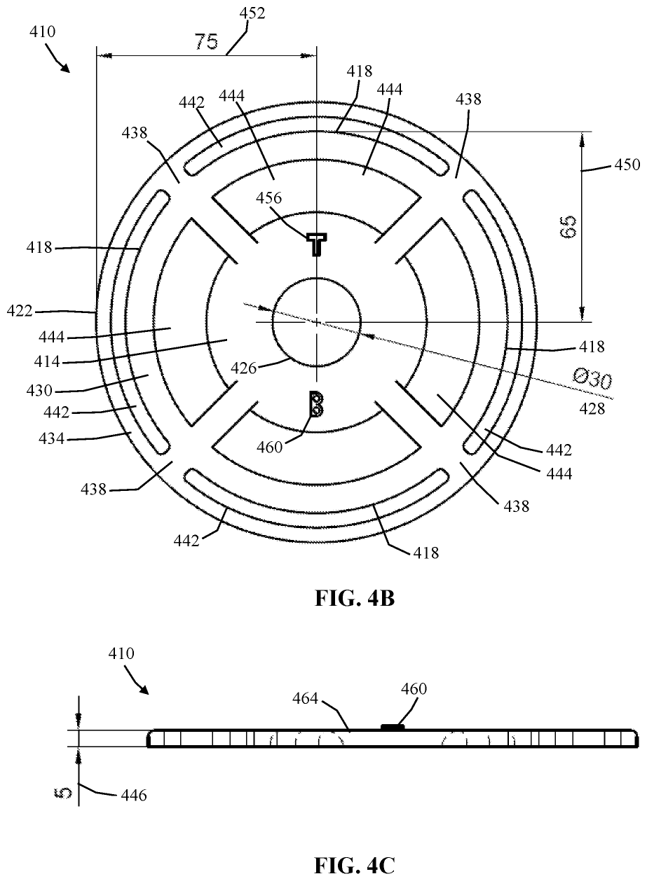

With reference to FIGS. 4A-4C, a template device 410 for positioning a breast implant at a desired location in a patient is illustrated. The device 410 includes a body 414 have a periphery 418 corresponding to an outer periphery of the breast implant. In the illustrated embodiment, the periphery 418 is a circumferential edge spaced radially inward from an outer-most circumferential edge 422 of the device 410. In the illustrated embodiment, the body 414 is circular.

With reference to FIG. 4B, the template device 410 further includes an aperture 426 formed in the body 414. In the illustrated embodiment, the aperture 426 is positioned at a geometric center of the device 414 (as viewed from FIG. 4B). The aperture 426 has a diameter 428 of approximately 30 cm.

In the illustrated embodiment, the body 414 includes a radially inward portion 430, a radially outward portion 434, and at least one strut 438 extending between the radially inward portion 430 and the radially outward portion 434. The body 414 includes a slot 442 at least partially formed between the radially inward portion 430, the radially outward portion 434, and the strut 438. In some embodiments, the body 414 includes a plurality of circumferential slots 442. In the illustrated embodiment, the body 414 includes four struts 438 extending between the radially inward and radially outward portions 430, 434 forming four slots 442. As such, the edge forming the periphery 418 corresponding to the breast implant shape is at least partially defined by one or more of the slots 442. The body 414 further includes four slots 444 positioned radially inward from the slots 442. The slots 444 can provide, for example, an area to grasp the device 410 with a surgical tool or hand.

In the illustrated embodiment, the periphery 418 is positioned at a prescribed radius 450 from the center aperture 426. In the illustrated embodiment, the radius 450 is approximately 65 cm. The outer-most circumferential edge 422 is positioned at a radius 452 from the center aperture 426. In the illustrated embodiment, the radius 452 is approximately 75 cm.

With reference to FIG. 4C, the body 414 has a thickness dimension 446 of approximately 5 cm. In some embodiments, the thickness dimension 446 is within a range of approximately 2 cm and approximately 6 cm.

The device 410 is configured to be positioned at the desired location in the patient and at least a portion of the periphery 418 is marked on the patient to identify the desired location for the breast implant.

As such, the template device 410 is a substantially flat device having a disc shape. The implant template device 410 has a circumferential slot or slots 442, which are disposed around the periphery of the template at the prescribed radius 450. In an example embodiment, an implant template device is unique to a particular breast implant size. Because breast implant sizes are typically provided in small increments (e.g., 0.5 cm), and to reduce the possibility of confusion, the template device can comprise one circumferential slot at an appropriate radius for a particular implant. That is, the implant template can be customized for each specific size and model of implant.

In some embodiments, the radially outward portion 424 includes a rim around the outside edge. In some embodiments, the rim is flexible but firm and maintains its general shape. In other embodiments, the rim is flexible and malleable such that it can be bent into any desired configuration. In some embodiments, the body 414 is trimmable to adjust the periphery and/or outermost edge of the body 414. The medical professional can alter or manipulate the device using any means known in the art (e.g., medical scissors), or the body can be perforated or pre-cut into various shapes, such that a separate tool is not necessary to configure the device for a particular subject. In this manner, the template device of the present disclosure is customizable for each subject. In some embodiments, the body further includes a second periphery corresponding to an outer periphery of a second breast implant. In other words, different edges formed on a single template device can correspond to different breast implants sizes and/or shapes. In some embodiments, the template device 410 is sterile, disposable, and foldable.

In some embodiments, the body 414 further includes a fiducial marking to indicate an orientation of the template device 410. In some embodiments, the fiducial marking is tactile. In the illustrated embodiment, the device 410 includes a first marking 456 of a raised “T” and a second marking 460 of a raised “B”. In the illustrated embodiment, the markings 456, 460 are formed on a surface 464 of the radially inward portion 430.

As such, the template device 410 provides a direct to implant placement guide that advantageously allows faster direct-to-implant positioning and placement. The template device 410 allows a surgeon to draw an outline of the breast implant without directly referencing or otherwise contaminating the breast implant. In some embodiments, the template devices are module, flat, circular designs with embed grooves or rounded edges that allow surgeons to draw an outline of a breast implant. Details of the template devices for positioning a tissue expander (e.g., materials, size, shape, spacing, coatings, etc.) apply equally to some embodiments of the template device for positioning a breast implant

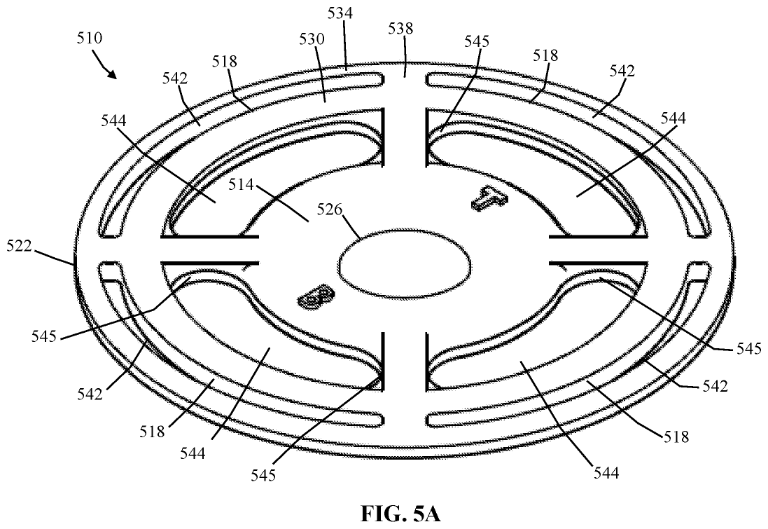

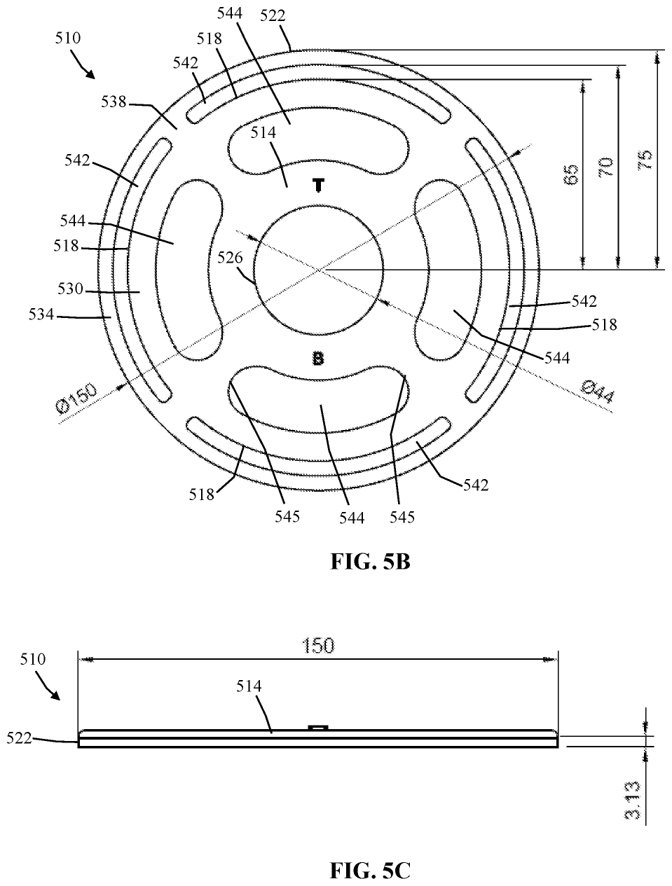

With reference to FIGS. 5A-5C, a template device 510 for positioning a breast implant at a desired location in a patient is illustrated. The template device 510 is similar to the template device 410, with similar aspects using similar reference numerals incremented by 100.

The device 510 includes a body 514 have a periphery 518 corresponding to an outer periphery of the breast implant. In the illustrated embodiment, the periphery 518 is a circumferential edge spaced radially inward from an outer-most circumferential edge 522 of the device 510.

With reference to FIG. 5B, the template device 510 further includes an aperture 526 formed in the body 514. In the illustrated embodiment, the aperture 526 is positioned at a geometric center of the device 514 (as viewed from FIG. 5B).

The body 514 includes a slot 542 at least partially formed between a radially inward portion 530, a radially outward portion 534, and struts 538. The body 514 further includes slots 544 positioned radially inward from the slots 542. In the illustrated embodiment, the slots 544 have arcuate ends 545. As such, the edge forming the periphery 518 corresponding to the breast implant shape is at least partially defined by one or more of the slots 542.

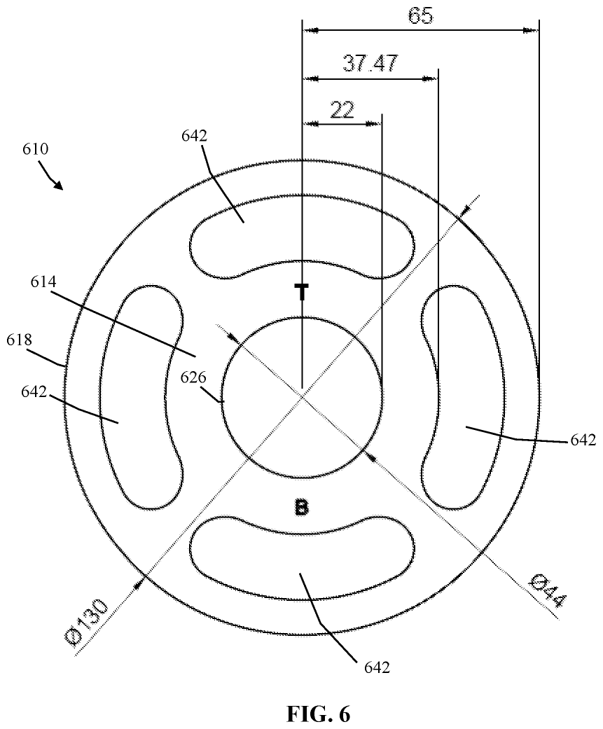

With reference to FIG. 6, a template device 610 for positioning a breast implant at a desired location in a patient is illustrated. The template device 410 is similar to the template device 410, with similar aspects using similar reference numerals incremented by 200. The device 610 includes a body 614 have a periphery 618 corresponding to an outer periphery of the breast implant. In the illustrated embodiment, the periphery 618 is the outer-most circumferential edge of the device 610. The device 610 further includes a center aperture 626 and four circumferential slots 642.

### 4. Kit

In accordance with the above embodiments, the present disclosure also provides a system or kit for securing a body implant in a desired position. In some embodiments, the system or kit includes any of the template devices described herein, and one or more of a body implant. Other components of the system or kit can also be included, as would be recognized by one of ordinary skill in the art based on the present disclosure, including but not limiting to, instructions for use, an adhesive agent, a sterilizing agent, a silicone disc, biodegradable attachment means (e.g., sutures), a biodegradable flexible tab, a placement tool, and a means for altering the shape of the device (e.g., scissors). and the like.

In one embodiment, a kit includes an implant and a template corresponding to the implant. The template is configured to mark a desired location in a patient prior to positioning the implant at the desired location. In some embodiments, the kit further includes a marker. In some embodiments, the implant is a tissue expander or a breast implant. In some embodiments, the kit further includes a biodegradable attachment means for securing the implant at the desired location.

### 5. Methods of Use

Another aspect of the present disclosure provides a method of positioning an implant or tissue expander (for simplicity, “implant”) using a template device as described herein. After the practitioner prepares an incision for the implant, the template device is inserted in the recess or possibly placed on the outer skin. The recess is typically above or below the pectoralis major. The template device can be rolled or folded to fit into a small incision and then opened once in the recess. The practitioner will then position the template device in the desired location and use a disposable, biocompatible marker, for example, to color in the apertures (e.g., holes, slots, etc.) of the template device, thus marking the patient's body. The template device is then removed and discarded. Afterwards, the implant is inserted and attached to the patient's body using the marked locations as a guide.

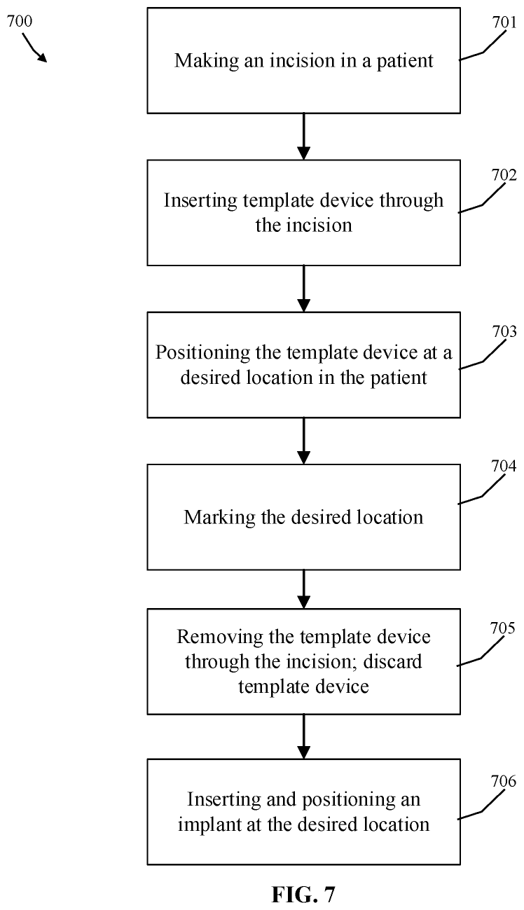

With reference to FIG. 7, a method 700 of marking a desired location for an implant in a patient is illustrated. The method 700 includes (STEP 701) making an incision in the patient; and (STEP 702) inserting a template (e.g., any one of the template devices disclosed herein) through the incision. In some embodiments, STEP 702 of inserting the template through the incision further includes folding the template to fit through the incision and unfolding the template once the template has passed through the incision.

The method 700 further includes (STEP 703) positioning the template in the desired location in the patient: (STEP 704) marking the desired location; and (STEP 705) removing the template through the incision. In some embodiments, STEP 704 of marking the desired location includes tracing a portion of the template (e.g., an aperture, a periphery, etc.) with a marker. In some embodiments, STEP 705 further includes discarding the template. In some embodiments, the method 700 further comprises (STEP 706) inserting an implant through the incision and positioning the implant at the desired location. In some embodiments, the method further includes attaching the implant to the patient at the desired location with attachment means.

One skilled in the art will readily appreciate that the present disclosure is well adapted to carry out the objects and obtain the ends and advantages mentioned, as well as those inherent therein. The present disclosure described herein are presently representative of preferred embodiments, are exemplary, and are not intended as limitations on the scope of the present disclosure. Changes therein and other uses will occur to those skilled in the art which are encompassed within the spirit of the present disclosure as defined by the scope of the claims.

No admission is made that any reference, including any non-patent or patent document cited in this specification, constitutes prior art. In particular, it will be understood that, unless otherwise stated, reference to any document herein does not constitute an admission that any of these documents forms part of the common general knowledge in the art in the United States or in any other country. Any discussion of the references states what their authors assert, and the applicant reserves the right to challenge the accuracy and pertinence of any of the documents cited herein. All references cited herein are fully incorporated by reference, unless explicitly indicated otherwise. The present disclosure shall control in the event there are any disparities between any definitions and/or description found in the cited references.

1. A device for positioning a tissue expander in a patient, the device comprising:
a body;
a first projection extending from the body;
a second projection extending from the body;
a first aperture positioned on the first projection;
a second aperture positioned on the second projection;
wherein the second aperture is positioned a first distance from the first aperture; wherein the first distance is equal to a second distance between attachment points on the tissue expander.

2. The device of claim 1, further comprising a third projection extending from the body and a third aperture position on the third projection; wherein the second projection is positioned between the first projection and the third projection; and wherein the third aperture is positioned a second distance from the second aperture.

3. (canceled)

4. (canceled)

5. The device of claim 2, wherein the second distance is larger than the first distance.

6. The device of claim 2, wherein the second distance is equal to the first distance.

7. The device of claim 1, further comprising a plurality of projections including the first projection and the second projection, and further comprising a plurality of apertures including the first aperture and the second aperture; wherein each of the plurality of projections includes at least one of the plurality of apertures.

8. The device of claim 7, wherein the plurality of apertures is equally spaced circumferentially around the body.

9. The device of claim 7, wherein the plurality of apertures is unequally spaced circumferentially around the body.

10. The device of claim 7, wherein the device is star shaped.

11. The device of claim 1, wherein the body includes a body aperture, and wherein the body aperture is positioned at a geometric center of the device.

12. The device of claim 1, wherein the device is flexible; and wherein the device is configured to fold for insertion through an incision in the patient and configured to conform to the contours of a chest wall of the patient.

13. (canceled)

14. The device of claim 1, wherein the device is silicone; and wherein the device is sterile, disposable, and foldable.

15. (canceled)

16. A device for positioning a breast implant at a desired location in a patient, the device comprising:
a body having a periphery corresponding to an outer periphery of the breast implant; and
an aperture formed in the body, the aperture positioned at a geometric center of the device; wherein the device is configured to be positioned at the desired location and the periphery is marked to identify the desired location for the breast implant.

17. The device of claim 16, wherein the periphery is an outer-most edge of the device.

18. The device of claim 16, wherein the periphery is an edge spaced radially inward from an outer-most edge of the device.

19. (canceled)

20. The device of claim 16, wherein the body is circular; and wherein the device is sterile, disposable, and foldable; and wherein the body is trimmable to adjust the periphery of the body.

21. (canceled)

22. The device of claim 16, wherein the body further comprising a fiducial marking to indicate an orientation of the device; and wherein the fiducial marking is tactile.

23. (canceled)

24. The device of claim 16, wherein the body includes a radially inward portion, a radially outward portion, and a strut extending between the radially inward portion and the radially outward portion; and wherein the body further includes a slot at least partially formed between the radially inward portion, the radially outward portion, and the strut; and wherein the periphery is at least partially defined by the slot.

25. (canceled)

26. (canceled)

27. The device of claim 16, wherein the body further includes a plurality of circumferential slots; wherein the body has a thickness within a range of 2 cm and 6 cm; and wherein the body further includes a second periphery corresponding to an outer periphery of a second breast implant.

28. (canceled)

29. (canceled)

30. A kit comprising:
an implant; and
a template corresponding to the implant, wherein the template is configured to mark a desired location in a patient prior to positioning the implant at the desired location.

31. The kit of claim 30, wherein the kit further includes a marker; and wherein the implant is a tissue expander or a breast implant; and wherein the kit further includes a biodegradable attachment means for securing the implant at the desired location.

32.-39. (canceled)
